(19) United States
(12) Patent Application Publication
(10) Pub. No.: US 2005/0228374 A1
(43) Pub. Date: Oct. 13, 2005

(54) Therapy apparatus for thermal sclerosing of body tissue

(76) Inventors: Kai Desinger, Berlin (DE); Markus Fay, Berlin (DE); Andre Roggan, Berlin (DE); Rainer Rothe, Lichtenau (DE)

Correspondence Address: BECK AND TYSVER P.L.L.C., 2900 THOMAS AVENUE SOUTH, SUITE 100, MINNEAPOLIS, MN, 55416, US

(21) Appl. No.: 10/515,867

(22) PCT Filed: May 20, 2003
(86) PCT No.: PCT/EP03/05303

§ 371 Date: May 27, 2005

(30) Foreign Application Priority Data
May 28, 2002 (DE) 10224153.8

(51) Int. Cl. A61B 18/18

(52) U.S. Cl. 606/45

(57) ABSTRACT

The application arrangement according to the invention for applying a high frequency current for thermal sclerosing of body tissue, includes:
an electrode needle 1 having an electrically conducting shaft 3,
at least one insulating casing body 7 which surrounds the electrically conducting shaft 3 and which is displaceable relative to the shaft 3 and which has a distal end from which the shaft 3 can be extended, and
at least one trocar 5 having a portion intended to be introduced into body tissue, a portion 11 intended to remain outside the body tissue, and a lumen 13 which extends through both portions and through which the shaft 3 of the electrode needle 1 is to be passed through the trocar 5.

The application arrangement according to the invention is distinguished in that the portion of the trocar 5 intended to be introduced into body tissue is electrically insulating and, in particular when the electrode needle or shaft 3 is passed through the lumen, forms the casing body 7 for the shaft 3 of the electrode needle 1.

The trocar 5 performs two functions. On the one hand it performs the conventional tasks of a trocar 5, more specifically for example permitting accurately targeted feed of drugs or removal of tissue and the introduction of electrode needles 1 without having to make a fresh puncture each time. On the other hand the part of the trocar 5 which is in the body serves as an insulating casing body 7 displaceable relative to the shaft 3 of the electrode needle 1 for adjusting the length of the active region of the shaft 3, that is to say that region which projects out of the insulating casing body 7 and is in electrically conductive contact with the body tissue when the electrode needle 1 is inserted into the body.

This application claims priority to PCT/EP03/05303, filed May 20, 2003 and to DE 102 24 153.8, filed May 27, 2002.

### FIELD OF THE INVENTION

The invention concerns a therapy apparatus, in particular an application arrangement for applying a high frequency current for the thermal sclerosing of body tissue.

### BACKGROUND OF THE INVENTION

Electrosurgical and in particular electrothermal sclerosing of pathologically altered body tissue is a method which is known in medicine. That method is of particular interest for the therapy of organ tumors, for example liver tumors. To perform the sclerosing procedure one or more electrodes are placed in the tissue to be sclerosed, that is to say the tumor tissue, or in the immediate proximity thereof, and an alternating current is caused to flow between the electrodes or an electrode and a so-called neutral electrode which is fixed externally to the body. When the current flows between the electrode and the neutral electrode (possibly also between a plurality of electrodes and one or more neutral electrodes), that is referred to as a monopolar electrode arrangement. If in contrast the current flows between the electrodes themselves disposed in the tissue (in that case at least two electrodes have to be introduced into the tissue), that is referred to as a bipolar arrangement.

To cause sclerosing of the pathologically altered tissue, a current flow is induced by means of a high frequency generator between the so-called active electrode which is in electrically conductive contact with the body tissue, and for example a neutral electrode. The electrical resistance of the body tissue in that respect provides that the alternating current is converted into heat. At temperatures between 50° C. and 100° C., that involves massive denaturing of the body-specific proteins and consequently causes the tissue area involved to die. By virtue of the high current density in the region of the active electrodes, heating of the tissue takes place predominantly where the active electrodes are in electrically conductive contact with the body tissue.

What is crucial for effective and in particular reliable therapy is the production of a thermal destruction zone which is optimally adapted to the extent of the pathological tissue. Here, the length of the non-insulated active region of the electrode needle places a decisive part. The longer that region is, the correspondingly greater is the axial extent of the thermal destruction zone.

The electrode intended for placement in the tissue is generally arranged on an electrode needle. An electrode needle is described for example in US No 2002/0035363. The electrode needle described therein includes an electrically conducting shaft and an insulating casing which is axially displaceable relative to the shaft. The active surface, that is to say the surface of the shaft which is to be brought into contact with the body tissue for use of the electrode needle, can be determined by displacement of the insulating casing.

In addition US No 2002/0035363 describes a trocar through which the electrode needle can be introduced into body tissue. A trocar is a body probe with a portion which is intended to be introduced into body tissue and a portion which is intended to remain outside the body tissue, as well as a free lumen for the introduction of instruments or for passing fluids in or out. It is used for example for discharging fluids from body cavities or introducing drugs in specifically targeted fashion into given regions of the body.

### SUMMARY OF THE INVENTION

An object of the invention is to provide an alternative application arrangement having a shaft and a casing body displaceable axially relative to the shaft, the application arrangement being of a simple structure.

A further object of the invention is to provide an application arrangement, in particular an electrode needle, comprising a shaft and a casing body displaceable axially relative to the shaft, which application arrangement can be used in a flexible manner.

The first object is attained by an application arrangement as set forth in claim 1 and the second object is attained by an application arrangement as set forth in claim 12.

In accordance with claim 1 there is provided an application arrangement for applying a high frequency current for thermal sclerosing of body tissue, including:

an electrode needle having an electrically conducting shaft,

at least one insulating casing body which surrounds the electrically conducting shaft and which is displaceable relative to the shaft and which has a distal end from which the shaft can be extended, and

at least one trocar having a portion intended to be introduced into body tissue, a portion intended to remain outside the body tissue, and a lumen which extends through both portions and through which the shaft of the electrode needle is to be passed through the trocar.

The application arrangement as set forth in claim 1 is distinguished in that the portion of the trocar intended to be introduced into body tissue is electrically insulating and, particularly in the case of a shaft or electrode needle which is passed through the lumen, forms the casing body for the shaft of the electrode needle.

The trocar performs two functions. On the one hand it performs the conventional tasks of a trocar, more specifically for example permitting accurately targeted feed of drugs or removal of tissue and the introduction of electrode needles without having to make a fresh puncture each time. On the other hand the part of the trocar which is in the body serves as an insulating casing body displaceable relative to the shaft of the electrode needle for adjusting the length of the active region of the shaft, that is to say that region which projects out of the insulating casing body and is in electrically conductive contact with the body tissue when the electrode needle is inserted into the body. An application arrangement of that kind is of a simplified structure in comparison with the state of the art in which, in addition to the trocar, there is a displaceable insulating casing around the shaft of the electrode needle. In particular, the application arrangement according to the invention also makes it possible to adjust the length of the active region of electrode needles which are not provided with their own insulating casing.

Because the trocar forms the casing body of the electrode needle and the latter therefore does not require its own casing body, the overall diameter of the portion of the trocar, which is intended for being introduced into the body tissue, can be kept small. Therefore puncturing with the application arrangement according to the invention is less traumatic than with the trocar-needle combination in accordance with the state of the art.

For displacement of the shaft relative to the casing body, that is to say relative to the trocar, there is provided a displacement device, for example using a guide element, with which the length of the part of the shaft which projects out of the distal end of the casing body can be adjusted. The displacement device can include in particular a clamping or screw mechanism for arresting the electrode needle relative to the casing body in order to counteract unintentional displacement of the casing body relative to the electrode needle.

Particularly accurate adjustment of the length of the part of the shaft which projects out of the distal end of the casing body can be achieved if the application arrangement has a guide element at the electrode needle, in particular at the proximal end of the shaft, and if there is provided a female and male screwthread combination on the trocar and on the guide element for axial displacement of the casing body relative to the shaft. The female and male screwthread combination makes it possible for the casing body to be displaced precisely relative to the shaft, by rotation of the guide element relative to the trocar. The accuracy of fine adjustment in such displacement can be established by a suitable choice in respect of the screwthread pitch. The smaller the screwthread pitch, the correspondingly smaller is the displacement for example in a full revolution of the screwthread, that is to say, the correspondingly more accurate can the fine adjustment be made.

In order to permit defined adjustment of the length of the part of the shaft which projects out of the casing body the electrode needle or the trocar, in particular the clamping or screw mechanism, can include markings from which it is possible to ascertain the length of the part of the shaft which projects out of the distal end of the casing body, when the shaft is introduced into the body. The markings permit specific targeted adjustment of the active length of the electrode, even when the electrode needle is introduced into the body.

In an embodiment of the invention the casing body is distinguished in that it closely embraces the shaft. Such close embrace prevents body fluid from penetrating between the shaft and the periphery of the casing. The result of body fluids penetrating in that way, as a conducting fluid, could be that not only the region of the shaft which projects out of the casing body and which therefore is not covered is in electrically conductive relationship with the body tissue, but also regions of the shaft which should actually be electrically insulated relative to the body tissue.

In a further embodiment of the invention there is a gap between the shaft and the inside of the casing body. In addition the portion of the trocar which is intended to remain outside the body can have a fluid feed for feeding fluids into the gap. When the electrode needle is inserted, the gap makes it possible for fluids, in particular liquids, to be introduced into the target area of the body tissue. Liquids which can be introduced into the target area are for example drugs, painkillers, flushing agents or liquids which counteract drying-out of the tissue during the application of the high frequency current and thus considerably enhance the efficiency of thermal destruction. In the latter case the liquids are preferably electrically conductive in order to maintain electrical contact of the shaft with the body tissue. For example physiological saline solutions present themselves as electrically conductive liquids.

The dimension of the gap can advantageously be so selected that a given liquid pressure has to be exceeded so that the liquid can flow through the gap. That configuration makes it possible to prevent electrically conducting body fluids from penetrating into the gap and thus forming an electrically conductive connection between the body tissue and parts of the shaft, which should actually be insulated by the casing body relative to the body tissue.

A further configuration of the invention is distinguished in that the shaft is provided with a point at its distal end. The point which, when the shaft is inserted into the casing body, projects out of the distal end of the casing body, can serve as a puncturing agent upon introduction of the trocar into the body tissue so that the electrode needle can be used as an insertion aid for the trocar.

In order to simplify insertion of the application arrangement and in particular the transition between the shaft and the casing body into the body tissue, the casing body can be provided at its distal end with a bevel, that is to say a tapering portion, so that there is not a stepped transition between the casing body and the shaft.

In an advantageous configuration of the application arrangement the portion of the trocar intended to be introduced into body tissue includes a material which makes it visible in a computer-tomographic or nuclear magnetic resonance tomographic recording so that placement of the trocar can be controlled by means of computer tomography or nuclear magnetic resonance tomography. Such a material can be for example gold.

In accordance with claim 13, to attain the second object, there is provided an application arrangement for applying a high frequency current for thermal sclerosing of body tissue, including:

an electrode needle having an electrically conducting shaft, and

at least one insulating casing body which surrounds the electrically conducting shaft and which is displaceable relative to the shaft and having a distal end from which the shaft can be extended.

The application arrangement set forth in claim 13 is distinguished in that a counterpart electrode is arranged at the outside of the insulating casing body. In that respect the counterpart electrode is to be taken to mean any electrode which permits bipolar operation of the application arrangement. In particular the shaft and the counterpart electrode are electrically independent of each other, that is to say respective mutually independent electrical potentials, in particular electrical potentials produced by a high frequency generator, can be applied to the shaft and the counterpart electrode, so that a high frequency current flows between them.

An electrode needle of that kind can be operated both in a bipolar and also a monopolar mode. Displaceability of the casing body makes it possible for the flow of current through the body tissue to be treated to be influenced by virtue of the length of the shaft portion of the electrode needle, which projects from the casing body-and thus the effective shaft surface area which can be brought into electrically conducting contact with the body tissue-being varied.

The described counterpart electrode can be used even when the casing body is formed by a trocar. It is then disposed on the portion of the trocar, which is intended to be introduced into body tissue. Advantageously the trocar then has its own electrical connection for connecting a high frequency generator to the counterpart electrode. In that situation the electrical connection can be in the form of a plug contact, in relation to which there is a counterpart portion on a part of the electrode needle, which is not intended to be introduced into the body tissue, so that the trocar is to be connected to the high frequency generator by way of the electrode needle. It is particularly user-friendly if the plug contact and the counterpart portion are of such an arrangement and configuration that the connection of the plug contact to the counterpart portion occurs automatically upon introduction of the electrode needle into the trocar.

Further features and advantages of the invention are described hereinafter by means of the description of embodiments by way of example with reference to the accompanying drawings.

### BRIEF DESCRIPTION OF THE DRAWINGS

FIG. 1 is a perspective view showing the electrode needle and the trocar of a first embodiment of the application arrangement according to the invention,

FIG. 2 shows a perspective view of the embodiment of FIG. 1 with the electrode needle inserted into the trocar,

FIG. 3 shows a view in longitudinal section of the first embodiment,

FIG. 4 shows a view in longitudinal section of a second embodiment of the application arrangement according to the invention,

FIG. 5 shows a view in cross-section through the casing body and the shaft of a third embodiment of the application arrangement according to the invention,

FIG. 6 shows a view in cross-section through the casing body and the shaft of a fourth embodiment of the application arrangement according to the invention,

FIG. 7 shows a first treatment configuration using the application arrangement according to the invention,

FIG. 8 shows a second treatment configuration using the application arrangement according to the invention, and

FIGS. 9A-9C show an example of medical treatment with the application arrangement according to the invention.

### DETAILED DESCRIPTION OF PREFERRED EMBODIMENT (s)

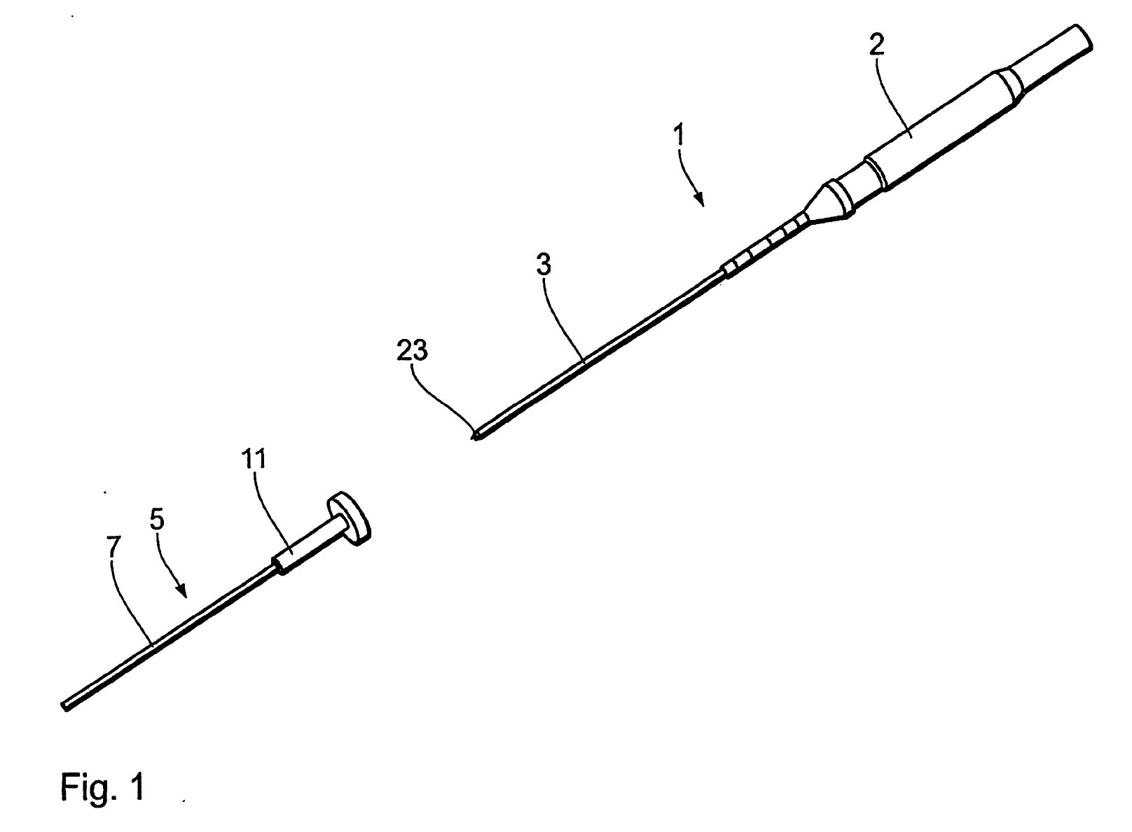

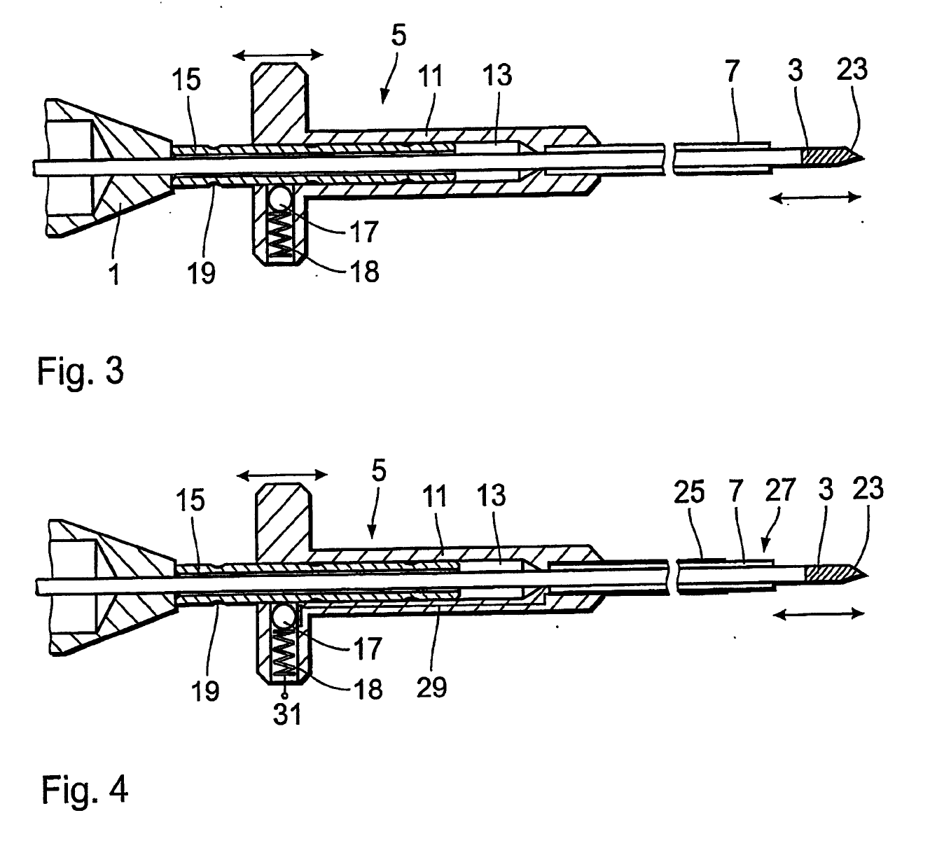

FIG. 1 shows a perspective view of a first embodiment of the application arrangement according to the invention. The application arrangement includes an electrode needle 1 with a gripping portion 2 for handling the electrode needle 1 and a shaft 3 of an electrically conductive material which can be connected to a high frequency generator (not shown). It further includes a trocar 5 having a portion 7 adapted to be introduced into body tissue and a portion 11 adapted to remain outside the body tissue. The trocar 5 has a lumen 13 (see FIG. 3) through which the electrode needle 1 can be introduced in such a way that the shaft 3 of the electrode needle 1 extends through the portion 7 of the trocar 5.

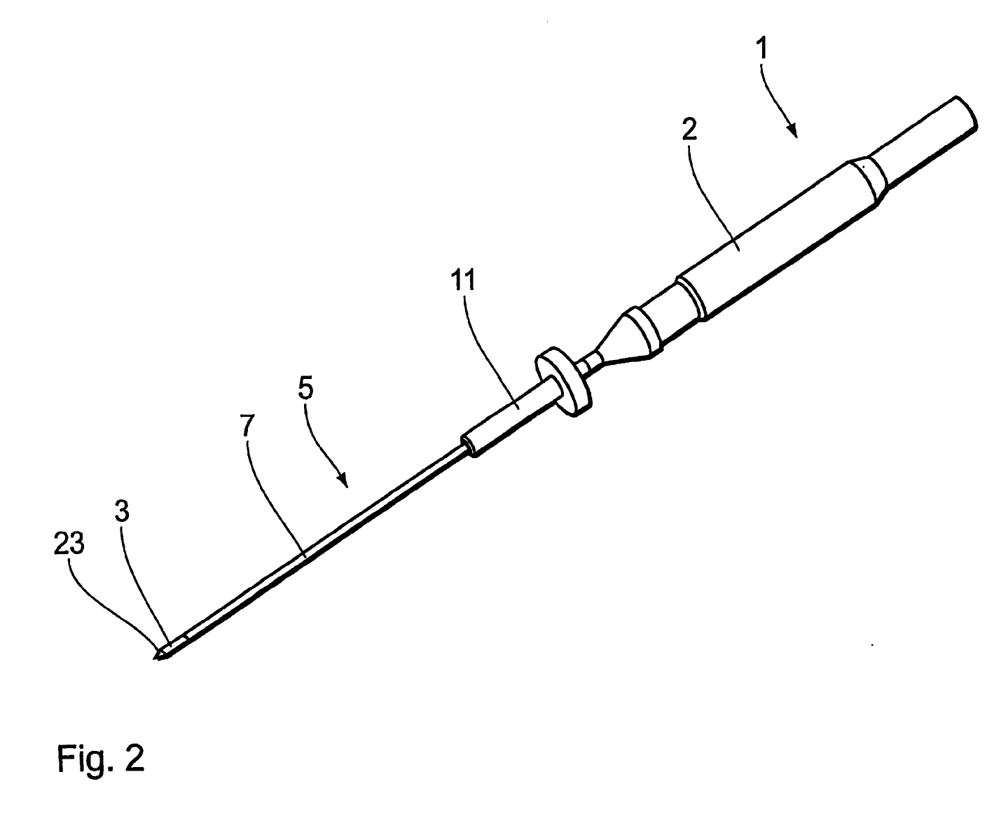

When the electrode needle 1 has been introduced into the trocar 5 (see FIG. 2) the portion 7 closely embraces the shaft 3 of the electrode needle 1. It comprises an insulating material so that it forms an insulating casing body for the shaft 3 of the electrode needle 1.

By means of a displacement device which, in the embodiment shown in FIG. 3, includes a cylindrical guide element 15, the electrode needle 1 which is introduced through the lumen 13 of the trocar 5 can be displaced axially relative to the trocar 5. In this case the displacement device includes markings which, in the present embodiment, are in the form of annular grooves 19 extending around the periphery of the guide element 15. Provided on the trocar 5 is a clamping mechanism 17 which co-operates with the displacement device 15 and which is in the form of a ball adapted for engagement into the annular grooves 19. The ball 17 is pressed against the peripheral surface of the displacement device by a prestressed compression spring 18 and can latch into the annular grooves 19 in order in that way to secure the displacement device to prevent unwanted axial displacement thereof. Instead of the annular grooves 19 it is also possible to use other arresting means if they permit latching engagement of the ball 17.

The length of the distal portion of the electrically conducting shaft 3, which projects out of the casing body 7, can be varied by displacement of the electrode needle 1 by means of the guide element 15 relative to the trocar 5 and thus relative to the casing body 7. In that case the portion of the shaft 3, which projects out of the distal end of the casing body 7, forms the active electrode of the electrode needle 1, that is to say the active electrode which is in contact with the body tissue after being introduced thereinto.

By way of a line (not shown) which extends in the interior of the shaft 3, it is connected to a high frequency generator by which a high frequency voltage can be applied to the active electrode. When the high frequency voltage is applied a counterpart electrode is placed against the outside of the body so that a high frequency current can flow between the shaft 3 and the counterpart electrode and results in destruction of the body tissue, for example tumor tissue. In that respect the shape and size of the destruction zone can be varied by the length of the portion of the shaft 3, which projects from the distal end of the casing body 7.

The shaft 3 of the electrode needle 1 can in addition also be used for introduction of the trocar 5 into the body tissue. For that purpose the shaft 3 has a point 23 at its distal end for puncturing the body tissue.

After application of the high frequency current the trocar 5 can remain in the body tissue, in which case then it is only the electrode needle 1 that is pulled out of the trocar 5. It can then be used for example for introducing drugs. The casing body 7 prevents entrainment of tumor cells when the electrode needle is withdrawn.

When the trocar 5 is withdrawn from the tissue after application of the high frequency current or possibly later, a fibrin adhesive can be introduced into the penetration passage when the trocar is withdrawn, in order to seal off the passage.

An alternative embodiment of the application arrangement is shown in FIG. 4. Components which do not differ from the embodiment illustrated in FIGS. 1 and 2 are denoted by the same references and are not described again hereinafter.

Unlike the embodiment shown in FIGS. 1 through 3 the application arrangement in FIG. 4 includes a further second electrode 25 serving as a counterpart electrode in relation to the shaft electrode formed by the shaft 3. It is arranged on the outer periphery of the electrically insulating casing body 7. The axial length of the counterpart electrode 25 is approximately 1-20 times the diameter of the shaft 3.

At the distal end of the casing body 7 extending around the entire periphery of the casing body 7 is an insulating portion 27 which insulates the counterpart electrode 25 from the shaft 3 and establishes the spacing of the counterpart electrode 25 from the shaft electrode. The counterpart electrode 25 has an electrical feed line 29 which is separate from the shaft 3 and which extends through the peripheral wall of the portion 11 of the trocar 5 and which, by way of the ball 17, in the preferred embodiment an electrically conductive ball, for example a metal ball, and the compression spring 18, is connected to a terminal 31 for the connection of a high frequency generator. Bipolar operation of the application arrangement is possible with that design configuration.

In the embodiment described with reference to FIG. 4 the casing body 7, instead of being a component part of the trocar 5, can also be a component part of the electrode needle 1, in which case the casing body 7 is still displaceable with respect to the shaft 3. An electrode needle of such a design configuration can also be used without a trocar. The presence of a trocar is therefore not necessary for varying the length of the part of the shaft 3 which projects from the casing body 7.

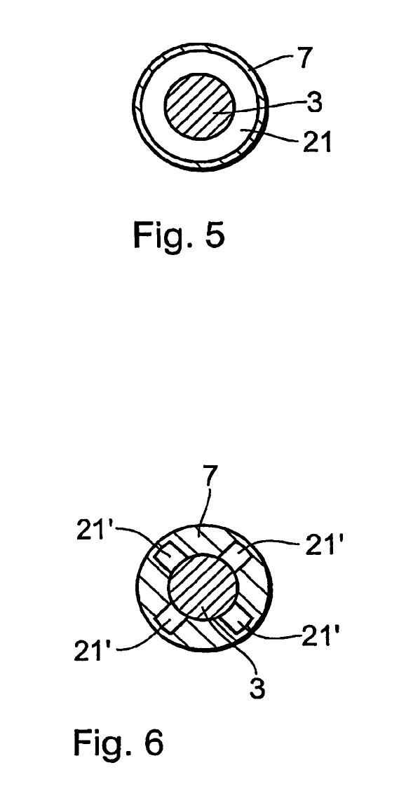

Further embodiments of the application arrangement according to the invention are shown in FIGS. 5 and 6 illustrating views in cross-section through the casing body 7 and the shaft 3 inserted into the lumen of the casing body 7. In the embodiment illustrated in FIG. 5 an annular gap 21 extends between the inside wall of the casing body 7 and the outside surface of the shaft 3, through which gap 21 for example a flushing fluid can be introduced into the tissue to be treated. In addition that gap 21 permits gases which are produced in the treatment procedure to escape from the body.

An alternative configuration is shown in FIG. 6. Instead of a gap which extends around the entire periphery of the shaft 3, this embodiment has four gaps 21′ which each extend only over a part of the periphery of the shaft 3. They are in the form of notches extending in the axial direction at the inside surface of the casing body 7 and extend over the entire length thereof. It will be appreciated that the number of notches can also be greater than or less than four. The notches also do not have to be of a quadrangular cross-section but for example can also be of a triangular or rounded cross-section.

The embodiment illustrated in FIG. 6 makes it possible for the casing body 7 to closely embrace the shaft 3 and thus, by virtue of its friction, to oppose displacement of the shaft with respect to the casing body 7. At the same time however fluids can be fed to the location to be treated in the body in the gaps 21′. Equally gases produced in the treatment can escape. The dimensions of the notches 21′ are preferably so selected that a predetermined fluid pressure has to be exceed so that the fluid can flow through the notches 21′. Body fluids which are mostly electrically conductive cannot then readily penetrate into the notches 21′.

For the introduction of fluids into the body tissue the trocar can have a bevel for a syringe connection, at its portion 11 which is adapted to remain outside the body. Instead of or in addition to the syringe connection the trocar can also have a lateral fluid feed means.

The electrode needle 1 and/or the portion 11 of the trocar which is intended to remain outside the body can also be provided with markings which show to the user how far the shaft 3 is projecting from the casing body 7. That is particularly important when the application arrangement is already disposed in the body so that the user does not have visual contact with the distal end of the casing body 7. Such markings can be afforded for example by the annular grooves 19 on the guide element 15, colored markings or a combination of the two.

To simplify the puncturing operation the casing body 7 can also be provided at its distal end with a bevel which is not illustrated in the Figures.

In an alternative embodiment the trocar 5 can also be sealed off to prevent the discharge of gases and body fluids. In that case the seal can be arranged for example in the form of an annular seal at the inside periphery of the portion 11 which is intended to remain outside the body, and can be of such a configuration that it can latch into the annular grooves 19 of the guide element 15 so that the seal is to be used at the same time as a latching element for arresting the electrode needle in an axial position relative to the trocar. The seal can alternatively also be arranged on the guide element, in which case the annular grooves are then arranged at the inside surface of the portion 11.

For the sake of better handling it is particularly desirable if the trocar 5 and/or the gripping portion 2 of the electrode needle 1 has grooving or knurling.

In a further configuration of the invention the trocar can have a plurality of axis-parallel portions provided for introduction into body tissue, as the casing bodies. The casing bodies each have a respective lumen which opens into a common lumen in the portion of the trocar which is intended to remain outside the body. Electrode needles having a plurality of shafts can be introduced into the body tissue through such a trocar. Advantageously, the casing bodies are displaceable separately from each other in relation to the respective shaft extending through them.

It is found that the use of different active lengths in respect of the shafts projecting out of the casing bodies, with electrodes which are introduced in approximately axis-parallel relationship, makes it possible to model the thermal destruction zone produced, within wide ranges. It is thus possible to achieve optimum adaptation of the destruction zone even when dealing with complex tumor geometries.

Admittedly, in the application arrangements illustrated with reference to the specific embodiments, there has been described a displacement device for displacement of the casing body relative to the shaft of the electrode needle, which has latching detent positions, but it is also possible to provide a displacement device with which it is possible to permit stepless displacement and arresting of the casing body relative to the shaft. In this respect it is possible for example to envisage a fixing screw which is provided in the trocar and with which the casing body can be securely fixed to the shaft in any relative position with respect to each other.

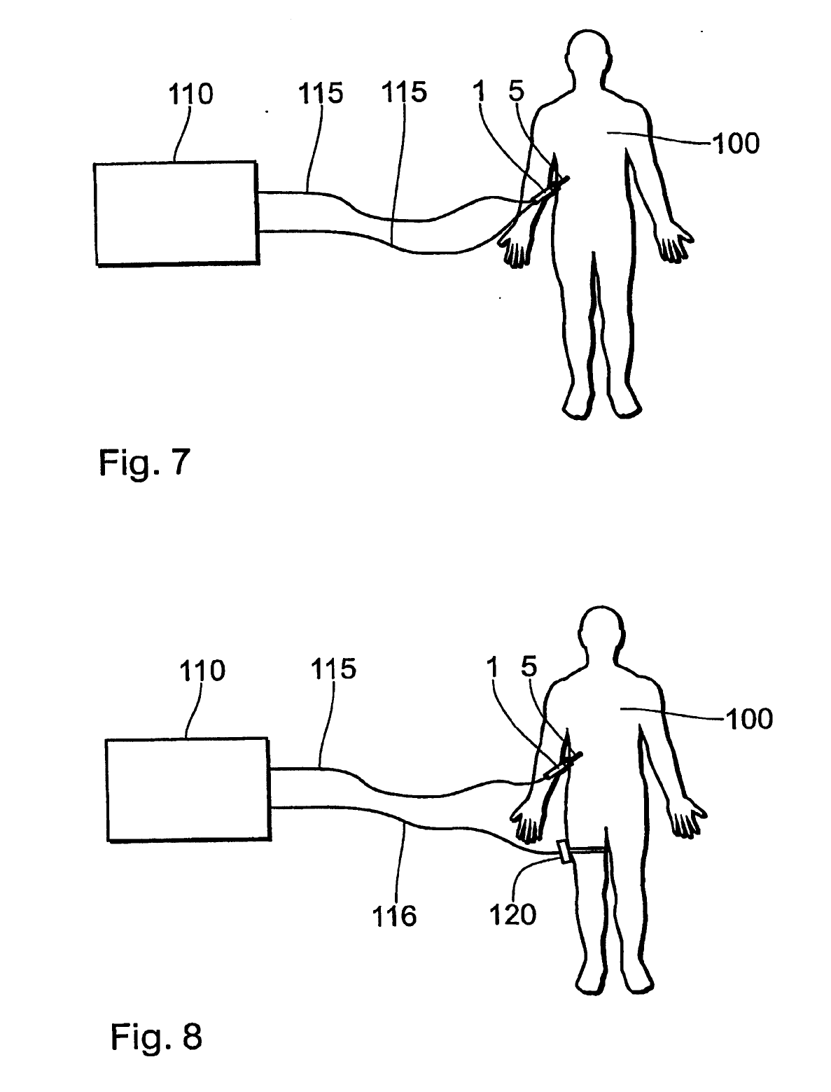

A first treatment configuration using the application arrangement according to the invention is diagrammatically shown in FIG. 7. An electrode needle 1 operated in the bipolar mode is introduced through a trocar 5 inserted into the body tissue of a patient 100, into a region of the body which is to be sclerosed, and connected to a high frequency generator 110 by way of two lines 115. The high frequency generator 110 provides the high frequency current which is necessary for the electrothermal sclerosing procedure and which flows through a circuit including the cables 115, the electrode needle 1 and the body tissue. In that case the high frequency current flows between the electrodes of the electrode needle 1 through the body tissue which is to be sclerosed.

A second treatment configuration using the application arrangement according to the invention is diagrammatically shown in FIG. 8. An electrode needle 1 which is operated in the monopolar mode is introduced through a trocar 5 inserted into the body tissue of a patient 100, into a region of the body which is to be sclerosed, and connected to a high frequency generator 110 by way of a line 115. In addition a neutral electrode 120 which is also connected to the high frequency generator 110 by way of a line 116 is fixed externally to the body of the patient 100. The high frequency generator 110 provides the high frequency current which is necessary for the electrothermal sclerosing procedure and which flows through a circuit including the cable 115, the cable 116, the electrode needle 1, the neutral electrode 120 and the body tissue. In that case the high frequency current flows between the electrode needle 1 and the neutral electrode through the body tissue which is to be sclerosed.

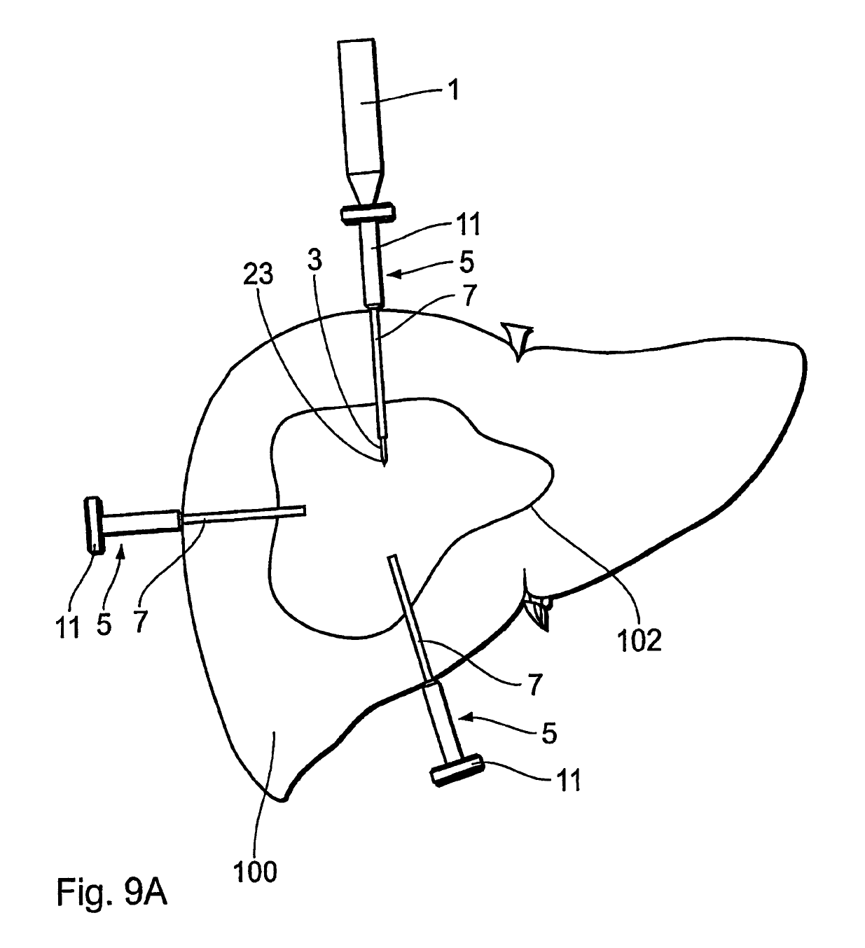

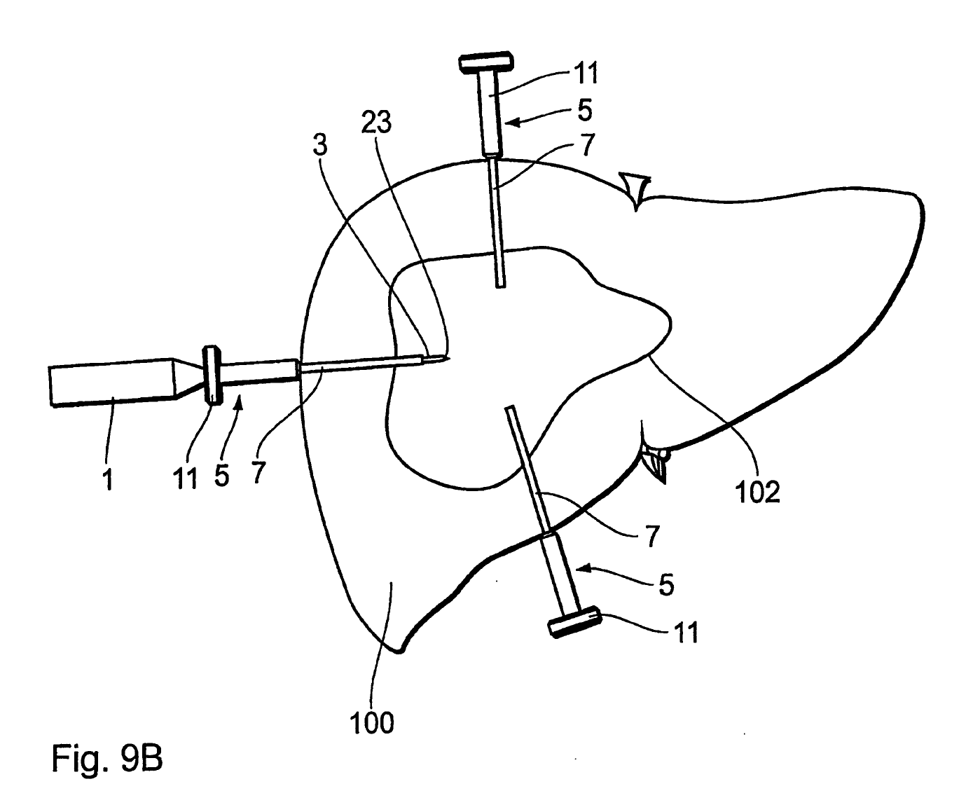

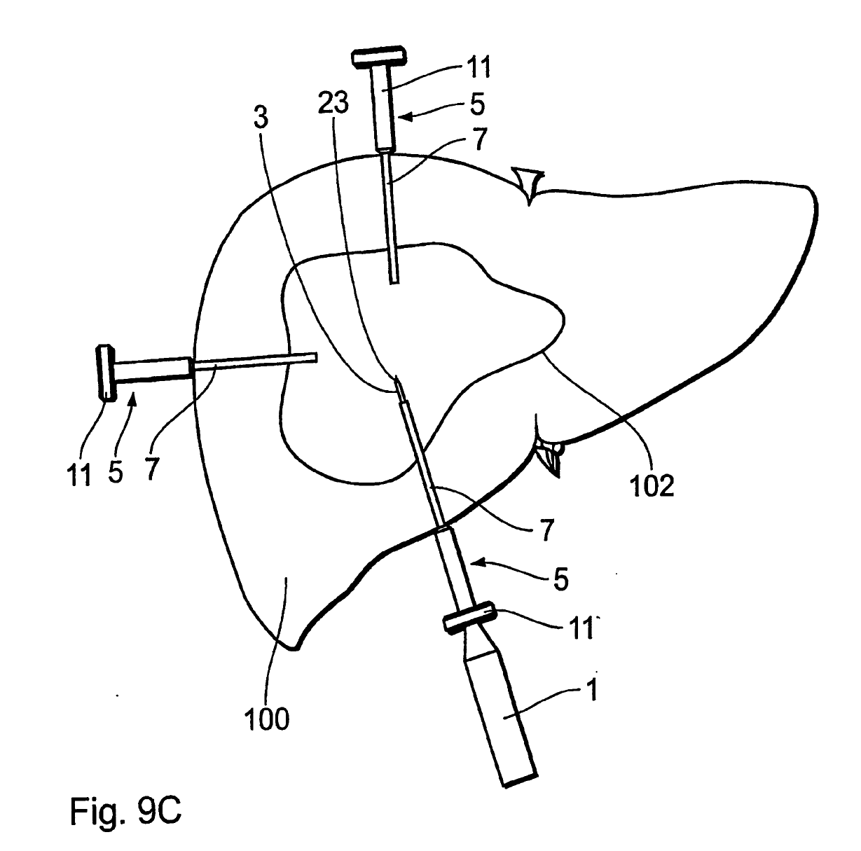

Reference will now be made to FIGS. 9A through 9C to describe a volume treatment procedure as an example of use of the application arrangement according to the invention. Volume treatment serves for the treatment of pathogenic tumor tissue, in particular in internal organs.

FIGS. 9A, 9B and 9C diagrammatically show a part of the body tissue 100 of a patient and tumor tissue 102 therein. For carrying out the volume treatment procedure a number of trocars 5 are introduced into the body of the patient in such a way that the distal ends of their portions provided for introduction into the body, that is to say the casing body 7, extend into the tumor tissue 102 or extend to same.

For introduction of a trocar 5 an electrode needle 1 is inserted with a point 23 at the distal end of its shaft 3 into the trocar 5 and that combination is introduced into the body tissue 100, the point 23 serving for puncturing purposes. All trocars 5 are placed in that fashion.

To destroy the tumor tissue 102 a neutral electrode (not shown in FIGS. 9A through 9C) is also fitted to the body of the patient, serving as a counterpart electrode in relation to the shaft 3 or, to put that better, the shaft electrode. When a high frequency voltage is applied to the electrode needle 1 a current then flows between the portion of the shaft 3, which projects from the distal end of the casing body 7 in the body tissue 100, and the neutral electrode. That causes destruction of the tumor tissue which is around the active shaft 3.

After a certain time or when a given degree of destruction of the tumor tissue is reached, the feed of the high frequency voltage is interrupted, the electrode needle 1 is withdrawn from the trocar 5 shown in FIG. 9A and introduced into the trocar 5 shown in FIG. 9B. There, application of the high frequency voltage is repeated in order in that way to destroy another portion of the tumor tissue 102. That is then repeated, as shown in FIG. 9C, in relation to a further trocar 5 which has been introduced into the body tissue 100.

Either all trocars 5 shown in FIGS. 9A through C can be introduced prior to the first application of the high frequency current, or alternatively each trocar 5 can be introduced immediately prior to the first application of the high frequency current at a given location, that is to say the trocars 5 used for applying the high frequency current in FIGS. 9B and 9C are only introduced immediately prior to the respective application step. In that case, a fresh trocar is introduced with each application of high frequency current at a location which has not yet been treated, until all trocars are fitted and the application operation only still takes place at locations which have already been treated previously and at which trocars are already disposed.

After application of the high frequency current the trocars 5 remain in the body region to be treated. They prevent the entrainment of tumor cells when the needle is withdrawn and serve for feeding drugs into the therapy volume, such as for example painkillers or chemical therapeutic agents. In addition, in the context of a fine needle biopsy, tissue sample can be taken from the target tissue, that is to say the tumor tissue, in order to perform histological analysis. After reintroduction of the electrode needle 1 into one of the trocars 5 application of the high frequency voltage can then be repeated. In that case on each occasion the length of the active region of the shaft 3 is adjusted in accordance with the clinical requirements.

As soon as the treatment is overall concluded, the trocars 5 are removed from the body tissue 100 again.

Alternatively electrode needles 1 can also be introduced simultaneously into all trocars 5 shown in FIGS. 9A through 9C and operated simultaneously. In that case the electrode needles 1 can be for example at a uniform potential, in which case the current then flows to one or more neutral electrodes (monopolar mode). Alternatively a multipolar mode of operation is also possible, that is to say a mode of operation in which the electrodes are operated at different potentials. Simultaneous operation of the electrode needles 1 permits an increase in efficiency, on the basis of the superposition principle, and that makes it possible to treat large tumors.

FIGS. 9A through 9C each show 3 trocars. The number of trocars used in the treatment however is not fixed at three but rather it can be adapted to the nature of the treatment and the nature and/or size of the tumor.

1. An application arrangement for applying a high frequency current for thermal sclerosing of body tissue, including:
an electrode needle having an electrically conducting shaft,
at least one insulating casing body which surrounds the electrically conducting shaft and which is displaceable relative to the shaft and which has a distal end from which the shaft can be extended, and
at least one trocar having a portion intended to be introduced into body tissue, a portion intended to remain outside the body, and a lumen which extends through both portions and through which the shaft of the electrode needle is to be passed through the trocar,
characterized in that
the portion of the trocar intended to be introduced into body tissue is electrically insulating and forms the casing body for the shaft of the electrode needle.

2. An application arrangement as set forth in claim 1 characterized by a displacement device for displacement of the casing body relative to the shaft in such a way that the length of the part of the shaft which projects from the distal end of the casing body is adjustable.

3. An application arrangement as set forth in claim 2 characterized by a clamping or screw mechanism for arresting the electrode needle relative to the casing body.

4. An application arrangement as set forth in claim 3 characterized in that the clamping or screw mechanism includes markings from which it is possible to ascertain the length of the part of the shaft which projects from the distal end of the casing body when the shaft is introduced into the body.

5. An application arrangement as set forth in claim 2 characterized in that provided on the portion, which is intended to remain outside the body of the trocar and the electrode needle is a female and male screwthread combination, the screwthreads of which are of such a configuration and relative arrangement that the casing body is to be displaced axially relative to the shaft by rotation of the electrode needle relative to the portion of the trocar which is intended to remain outside the body.

6. An application arrangement as set forth in claim 1 characterized in that the casing body closely embraces the shaft.

7. An application arrangement as set forth in claim 1 characterized in that there is a gap between the shaft and the inside of the casing body.

8. An application arrangement as set forth in claim 7 characterized in that the dimension of the gap is so selected that a given fluid pressure has to be exceeded so that fluid can flow through the gap.

9. An application arrangement as set forth in claim 7 characterized in that a fluid feed for feeding fluids into the gap is arranged on the portion of the trocar, which is intended to remain outside the body.

10. An application arrangement as set forth in claim 1 characterized in that at its distal end the shaft is provided with a point.

11. An application arrangement as set forth in claim 10 characterized in that at its distal end the casing body has a bevel.

12. An application arrangement as set forth in claim 1 characterized in that the portion of the trocar which is intended to be introduced into body tissue includes a material which makes it visible in a computer tomographic or nuclear magnetic resonance tomographic recording.

13. An application arrangement for applying a high frequency current for thermal sclerosing of body tissue, including:
an electrode needle having an electrically conducting shaft,
at least one insulating casing body which surrounds the electrically conducting shaft and which is displaceable relative to the shaft and having a distal end from which the shaft can be extended,
characterized in that
a counterpart electrode is arranged at the outside of the insulating casing body.

14. An application arrangement as set forth in claim 1 comprising a counterpart electrode arranged at the outside of the insulating casing body.

15. An application arrangement as set forth in claim 14 characterized in that the trocar has an electrical connection for connecting a high frequency generator to the counterpart electrode.

16. An application arrangement as set forth in claim 15 characterized in that the electrical connection is in the form of a plug contact, in relation to which there is a counterpart portion on a part of the electrode needle, which is not intended for being introduced into the body tissue.

17. An application arrangement as set forth in claim 16 characterized in that the plug contact and the counterpart portion are of such a configuration and relative arrangement that the connection of the plug contact to the counterpart portion is produced automatically when the electrode needle is introduced into the trocar.
